SEQUENCE LISTING (16 sequences)

SEQ ID NO: 1 - protein, 471 aa, Artificial Sequence
  Heavy chain for humanized GHR106
MDPKGSLSWRILLFLSLAFELSYGQVQLQESGPGLVKPSETLSLTCTVSGFSLSRYSVHWIRQPPGKGLEWIGMIWGGGSTDYNPSLKSRVTISKDNSKSQVFLKMSSVTAADTAMYYCARGNDGYYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 1380 bp, Artificial Sequence
  Nucleotide sequence of humanized GHR-106 heavy chain
atggaccccaagggcagcctgagctggagaatcctgctgttcctgagcctggccttcgagctgagctacggccaggttcagttgcaagagtctggtcccggcctggttaaaccctctgagactttgagcttgacatgcaccgtaagcggcttctctctgagccggtactctgtacactggattcgacagcctcctggcaagggcctggaatggattggcatgatctggggcggcggaagtacagattataacccttccttgaagagccgcgtcaccatctctaaggacaacagcaagtcccaggtcttcctgaagatgtcaagcgtcaccgctgccgacaccgccatgtactactgtgcacgggggaatgacggatactacagtttcgcatattggggccagggaacactggttacagtgtctagtgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccac

SEQ ID NO: 3 - protein, 239 aa, Artificial Sequence
  Light chain of humanized GHR-106 antibody
METDTLLLWVLLLWVPGSTGDIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 720 bp, Artificial Sequence
  Nucleotide sequence of light chain of humanized GHR-106 antibody
atggagaccgacaccctgctgctctgggtgctgctgctctgggtgcccggctccaccggagatatcgtaatgactcagtcccccgatagcctcgctgtgtcattgggtgaacgggcaactattaactgtaagtcatcacaaagcctccttaattctaggaccaggaaaaactacctggcatggtatcaacagaagccaggacagtcaccaaagctgctgatctactgggcttctacaagagagagtggagtgccagaccgcttctccggctccgggagcggcactgattttaccctcactatcagctcccttcaggcagaggatgtggccgtgtactattgcaagcagagctacaacctctacaccttcggccaggggactaaactggaaattaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 5 - protein, 118 aa, Artificial Sequence
  VH sequence of humanized GHR-106 antibody
QVQLQESGPGLVKPSETLSLTCTVSGFSLSRYSVHWIRQPPGKGLEWIGMIWGGGSTDYNPSLKSRVTISKDNSKSQVFLKMSSVTAADTAMYYCARGNDGYYSFAYWGQGTLVTVSS

SEQ ID NO: 6 - protein, 112 aa, Artificial Sequence
  VL sequence of humanized GHR-106 antibody
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLYTFGQGTKLEIK

SEQ ID NO: 7 - protein, 687 aa, Artificial Sequence
  Amino acid sequence of GHR-106 CAR fusion protein construct
AATMALPVTALLLPLALLLHAARPQVQLQESGPGLVKPSETLSLTCTVSGFSLSRYSVHWIRQPPGKGLEWIGMIWGGGSTDYNPSLKSRVTISKDNSKSQVFLKMSSVTAADTAMYYCARGNDGYYSFAYWGQGTLVTVSSGGGGSGGGGSGGGGSDIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 8 - DNA, 20 bp, Artificial Sequence
  Primer
ggcactgacaattccgtggt

SEQ ID NO: 9 - DNA, 20 bp, Artificial Sequence
  Primer
agggacgtagcagaaggacg

SEQ ID NO: 10 - DNA, 23 bp, Artificial Sequence
  Probe
acgtcctttccatggctgctcgc

SEQ ID NO: 11 - DNA, 22 bp, Artificial Sequence
  Primer
gctgtcatctcttgtgggctgt

SEQ ID NO: 12 - DNA, 21 bp, Artificial Sequence
  Primer
actcatgggagctgctggttc

SEQ ID NO: 13 - DNA, 26 bp, Artificial Sequence
  Probe
cctgtcatgcccacacaaatctctcc

SEQ ID NO: 14 - DNA, 20 bp, Artificial Sequence
  Primer
tgacagccgcctagcatttc

SEQ ID NO: 15 - DNA, 24 bp, Artificial Sequence
  Primer
gctcgatatcagcagttcttgaag

SEQ ID NO: 16 - DNA, 22 bp, Artificial Sequence
  Probe
cacgtggcccgagagctgcatc

(19) United States
(12) Patent Application Publication
(10) Pub. No.: US 2022/0242960 A1
(43) Pub. Date: Aug. 4, 2022

(54) GHR-106 CHIMERIC ANTIGEN RECEPTOR CONSTRUCT AND METHODS OF MAKING AND USING SAME

(71) Applicant: Gregory Chi-Yu LEE, Vancouver (CA)

(72) Inventor: Gregory Chi-Yu LEE, Vancouver (CA)

(21) Appl. No.: 17/589,474

(22) Filed: Jan. 31, 2022

Related U.S. Application Data
(63) Continuation of application No. 16/474,936, filed on Jun. 28, 2019, now abandoned.
(60) Provisional application No. 62/480,229, filed on Mar. 31, 2017.
(60) Provisional application No. 62/441,380, filed on Jan. 1, 2017.

(51) Int. Cl. C07K 16/28; A61K 35/17; C07K 14/725; C12N 5/0783; C12N 15/62

(52) U.S. Cl. CPC C07K 16/2869; A61K 35/17; C07K 14/7051; C12N 5/0638; C12N 15/62; C07K 2317/24; C12N 2740/16043; C07K 2317/622; C07K 2317/73; C07K 2319/03; C07K 2319/33; C12N 2510/00; C07K 2317/53

(57) ABSTRACT

A chimeric antigen receptor (CAR) having an antigen binding domain capable of binding to extracellular domains of human GnRH receptor. The antigen binding domain can have a binding affinity and specificity similar to the GHR-106 antibody. Methods of making and using such CARs are provided. The CARs can be used to treat cancer.

### REFERENCE TO RELATED APPLICATIONS

This application claims priority to, and the benefit of, U.S. provisional patent application No. 62/441380 filed 1 Jan. 2017 and U.S. provisional patent application No. 62/480229 filed 31 Mar. 2017. Both of the foregoing applications are incorporated by reference herein for all purposes.

### TECHNICAL FIELD

Some embodiments of the present invention relate to the fields of immunology, cell biology, molecular biology, and medicine, including cancer medicine. Some embodiments of the present invention relate to the field of a chimeric antigen receptor (CAR) that targets gonadotropin-releasing hormone (GnRH) receptor, nucleotide constructs encoding such a CAR, and methods of making and using same.

### BACKGROUND

Gonadotropin releasing hormone (GnRH) receptors are located on the external membrane of selected cell types. Through specific binding to the GnRH receptor, the anterior pituitary releases GnRH, a decapeptide hormone that stimulates the release of gonadotropin, luteinizing hormone (LH) and follicle stimulating hormone (FSH). Studies reveal that GnRH or its analogs can have anti-proliferative effects on cancer cells (Pati, D., et al., Endocrin (1995) 136:75-84; Choi, K. C., et al., J Clin Endocrinol & Metab (2001) 86:5075-5078, both of which are incorporated by reference herein for all purposes). GnRH analogs have been used to treat different cancers in humans and disorders in fertility regulation (Gnananpragasam, V. J., et al., J Pathol (2005) 206: 205-213; So, W. K., et al., FEBS Journal (2008) 275: 5496-5511).

A shortcoming associated with GnRH and GnRH analogs is that they have a relatively short half-life in circulation. In contrast, monoclonal antibodies generally have a relatively long half-life in circulation.

The GHR-106 monoclonal antibody was generated in mice immunized against synthetic peptides corresponding to the extracellular domains of the human GnRH receptor. GHR-106 was found to behave as a GnRH analog. GHR-106 has a much longer half-life than other GnRH analogs known in the art. GnRH analogs have been known for decades to treat different cancers in humans as well as disorders in fertility regulation.

U.S. Pat. Nos. 8,163,283, 8,361,793, and 9,273,138, which are incorporated by reference herein in their entirety for all purposes, are of interest with respect to the subject matter described herein. The amino acid sequences for murine GHR-106 and humanized GHR-106 were disclosed in U.S. Pat. No. 9,273,138 to Lee. Biochemical and immunological experiments demonstrate that both murine GHR-106 and humanized GHR-106 antibodies have high specificity and affinity to the extracellular domains of the human GnRH receptor, these domains being expressed on the surface of cancer cells of many tissue origins. For example, U.S. Pat. No. 8,163,283 to Lee discloses that the extracellular domains of the human GnRH receptor are expressed on the surface of cancer cells originating from breast, cervix, colon, glioblastoma, hepatocellular, kidney, lung, lymphoma, leukemia, neuroblastoma, placenta, and prostate tissues.

Additionally, murine GHR-106 and humanized GHR-106 antibodies behave similarly to GnRH peptide analogs in that they can induce in vitro apoptosis in treated cancer cells. Furthermore, unlike GnRH peptide analogs, both the murine and humanized forms of GHR-106 can induce complement-dependent cytotoxicity in treated cancer cells.

Chimeric antigen receptors (CARs) are artificial receptors that convey antigen specificity to cells, such as T cells. CAR in T cell therapy (CAR-T) technology combines T cell immunotherapy, gene therapy and immunotherapy. CAR-T has been used for cancer treatments and it involves modifying a patient's T cells. The modified T cells express CARs, which are antigen receptors recognizing cell surface antigens on tumor cells. Upon antigen binding, the modified T cells can initiate an immune response, such as the release of cytokine to induce tumor cell death. Attempts in using CAR-T to treat cancer have met with some success. Successful examples have been reported for CAR-T c ell therapy of different types of blood cancers, for example by using a CD19-related CAR platform. U.S. Pat. No. 8,916,381, incorporated by reference herein in its entirety, discloses a method of treating leukemia with CAR-T. Brentjens et al. (Molecular Therapy: Treatment of Chronic Lymphocytic Leukemia with Genetically Targeted Autologous T Cells: Case Report of an Unforeseen Adverse Event in a Phase I Clinical Trial. 18 Vol. Elsevier, Apr. 1, 2010), incorporated by reference herein in its entirety, conducted a clinical trial to treat chronic lymphocytic leukemia with CAR-T. The modified T cells were designed to recognize CA19, which is expressed on most B-cell malignancies.

There remains a need for improved constructs and methods for selectively targeting and killing cancer cells.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a nucleotide vector capable of expressing a GHR-106 CAR. In some aspects, the GHR-106 CAR encodes a polypeptide having from N-terminal to C-terminal an antigen binding domain capable of binding to an extracellular domain of human GnRH receptor, a hinge domain, a transmembrane domain; and an intracellular T cell signaling domain. In some aspects, the antigen binding domain is an scFv of GHR-106.

One aspect of the invention provides a polypeptide that is a GHR-106 CAR that has an antigen binding domain capable of binding to the extracellular domains of human GnRH receptor. In some aspects, the polypeptide that is a GHR-106 CAR has an antigen binding domain capable of binding to an extracellular domain of human GnRH receptor, a transmembrane domain, and an intracellular T cell signaling domain. In some aspects, immune cells that express the GHR-106 CAR are able to selective bind to and kill cells expressing the human GnRH receptor. In some embodiments, the cells expressing the human GnRH receptor are cancer cells.

One aspect of the invention provides a method of producing an immune cell capable of expressing a GHR-106 CAR. The method involves isolating the immune cells from the subject and genetically engineering the immune cells to express a GHR-106 CAR. In some aspects, the genetic engineering can be carried out using a lentiviral vector. In some aspects, the immune cells are introduced into the body of a patient suffering from cancer or another disorder involving a high surface expression of human GnRH receptor to treat the cancer or the disorder.

In addition to the exemplary aspects and embodiments described above, further aspects and embodiments will become apparent by reference to the drawings and by study of the following detailed descriptions.

### BRIEF DESCRIPTION OF THE DRAWINGS

In drawings which illustrate non-limiting embodiments of the invention:

FIGS. 1A and 1B show amino acid and nucleotide sequences, respectively, of the heavy chain of a humanized GHR-106 antibody (SEQ ID NOS:1-2). FIGS. 1C-1D show the amino acid and nucleotide sequences, respectively, of the light chain of a humanized GHR-106 antibody (SEQ ID NOS:3-4). The sequences of the humanized GHR-106 antibody were deduced from antibodies produced by a stable cell line, UY464-GHR106.

FIG. 2A is a schematic diagram showing the domains of a first example embodiment of a nucleotide vector capable of expressing a GHR-106 CAR. The illustrated embodiment is a CAR lentiviral vector.

FIGS. 2B and 2C show amino acid sequences of the scFv fragment of the exemplary GHR-106 CAR nucleotide vector construct shown in FIG. 2A. FIG. 2B shows the amino acid sequence of the V_{H} domain of the scFv fragment (SEQ ID NO:5). FIG. 2C shows the amino acid sequence of the V_{L} domain of the scFv fragment (SEQ ID NO:6).

FIG. 2D shows the protein sequence of the exemplary GHR-106 CAR construct shown in FIG. 2A fused with an IL7 cytokine (SEQ ID NO:7). Although the sequences of FIG. 2D have been separated into different sections to illustrate the different domains of the GHR-106 CAR fusion protein construct, the sequences are one continuous polypeptide.

FIG. 3 is a schematic diagram showing schematically an example embodiment of a complete recombinant GHR-106 CAR-T lentiviral vector construct. The amino acid sequence encoded by the GHR-106 CAR-T nucleotide vector construct comprises SEQ ID NO:7. In this example embodiment, the gene sequence of the GHR-106 CAR nucleotide vector construct is present in the form of a plasmid that can be used as a transfer plasmid to produce lentivirus capable of introducing the nucleotide vector capable of expressing the GHR-106 CAR into an immune cell, e.g. a T cell.

FIG. 4 shows the molecular weights of DNA fragments produced by digesting the exemplary GHR-106 CAR nucleotide vector construct shown in FIG. 3 with restriction endonucleases EcoRI and XbaI.

FIGS. 5A and 5B show qPCR standard curves used to reveal titers by lentivirus titration. FIG. 5A shows the WPRE standard curve and FIG. 5B shows the ALB standard curve.

FIGS. 6A and 6B show validation of the insertion of the GHR-106 CAR nucleotide vector into transduced T cells by using a standard curve of Ct (cycle threshold) value to determine copy number. FIG. 6A shows the ALB standard curve and FIG. 6B shows the LTR standard curve. These two standard curves were used to determine the average number of copies of the exemplary GHR-106 CAR-T nucleotide vector construct in genetically modified T cells.

FIGS. 7A, 7B and 7C show the results of three repeats of a lysis assay, where genetically modified T cells comprising copies of the exemplary GHR-106 CAR nucleotide vector construct were co-cultured with tumor cells. Data for three repeats of CAR-T validation demonstrated by lysis of target tumor cells by GHR-106 CAR-T cells as measured by lactate dehydrogenase (LDH) assay are shown.

FIGS. 8A, 8B, 8C, 9A, 9B, 9C and 10A, 10B, 10C show standard curves for three separate repeats of the same experiment for IL-2, IFN-gamma and IL-7, respectively. FIGS. 8D, 8E, 8F, 9D, 9E, 9F and 10D, 10E, 10F show the level of cytokines (D=IL-2, E=IFN-gamma and F=IL-7, respectively) produced by genetically modified T cells comprising copies of the exemplary GHR-106 CAR-T nucleotide vector construct, when the genetically modified T cells were co-cultured with cervical tumor (C33A) cells.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term “nucleic acid molecule” refers to a polymeric form of nucleotides of any length. The nucleotides can include either ribonucleotides (RNA) or deoxyribonucleotides (DNA).

The terms “antibody” and “immunoglobulins” refer to antibodies of any isotype and fragments of antibodies that bind specifically to an antigen. Some examples of antibodies include but are not limited to humanized antibodies, chimeric antibodies, and proteins comprising an antigen-binding portion of an antibody.

The term “antibody fragment” refers to a portion of an antibody. Some examples of antibody fragments include but are not limited to an antigen binding (Fab) fragment, an F(ab')₂ fragment, an Fab' fragment, or a variable domain (Fv).

The term “single-chain variable fragment” (scFv fragment) refers to a single polypeptide chain, comprising the variable regions of the light (V_{L}) and heavy (V_{H}) chains of an antibody. The V_{L} and V_{H} regions are joined by a suitable linker.

The term “affinity” refers to the strength of the binding interaction between a single biomolecule and its ligand or binding partner. In some embodiments, the strength of the binding interaction is measured by the equilibrium constant for the reversible binding of two molecules, which can be expressed as a dissociation constant (K_{d}).

The terms “treat”, “treating” and “treatment” refer to an approach for obtaining desired clinical results. Desired clinical results can include, but are not limited to, reduction or alleviation of at least one symptom of a disease. For example, treatment can be diminishment of at least one symptom of disease, diminishment of extent of disease, stabilization of disease state, prevention of spread of disease, delay or slowing of disease progression, palliation of disease, diminishment of disease reoccurrence, remission of disease, prolonging survival with disease, or complete eradication of disease.

The terms “cancer cell” and “tumor cell” refer to cells, the growth and division of which can be typically characterized as unregulated. Cancer cells can be of any origin, including benign and malignant cancers, metastatic and non-metastatic cancers, and primary and secondary cancers.

The term “chimeric antigen receptor (CAR)” refers to an engineered receptor. A typical CAR has an antigen binding domain that binds to a desired target antigen, a transmembrane domain, and an intracytoplasmic domain. The antigen binding domain of a CAR can be provided by the scFv of a monoclonal antibody. The transmembrane domain and the intracytoplasmic domain can be provided by the CD3-zeta transmembrane and ectodomains. A typical CAR will also include a signal peptide at its amino-terminal end, to direct the nascent translated protein into the endoplasmic reticulum so that the antigen binding domain will be presented on the surface of the immune cell in which the CAR is expressed.

In some embodiments, a chimeric antigen receptor (CAR) comprising an antigen-binding fragment of a humanized GHR-106 monoclonal antibody is provided, and is referred to herein as a GHR-106 CAR. A GHR-106 CAR is a CAR that is able to bind to extracellular domains of the human GnRH receptor.

In some embodiments, the GHR-106 CAR comprises the scFv fragment of a humanized GHR-106 antibody, and can be expressed following introduction of a nucleotide vector encoding the GHR-106 CAR into suitable immune cells, for example, T cells. In some embodiments, the antigen binding domain of the GHR-106 CAR binds to the extracellular domains of human GnRH receptor. In some embodiments, the GHR-106 CAR exhibits similar binding affinity and specificity towards the human GnRH receptor as does a humanized GHR-106 antibody.

Some embodiments of the invention relate to the field of a lentiviral chimeric antigen receptor (CAR) nucleotide construct including portions of a humanized GHR-106 gene that provides a nucleotide vector capable of expressing a GHR-106 CAR in a transduced immune cell. Humanized GHR-106 is murine GHR-106 monoclonal antibody in humanized form which recognizes and binds specifically the extracellular domains of the human GnRH receptor. The human GnRH receptor is highly expressed on the surface of cancer cells of many tissue origins.

In some embodiments, upon transduction of a nucleotide vector encoding the GHR-106 CAR into suitable immune cells, for example, T cells or NK cells isolated from an individual patient, the GHR-106 CAR-transduced immune cells will then express a GHR-106 CAR construct with an antigen binding region comprising an scFv chain of a humanized GHR-106 antibody. This immunoglobulin chain will bind to surface-expressed GnRH receptor on cancer cells and result in cytotoxic killing of the cancer cells. The human GnRH receptor is expressed in particular on the surface of cancer cells in hormone-sensitive forms of cancer. Therefore, the GHR-106 CAR system can potentially be utilized for therapeutic applications for treatment of human hormone-sensitive cancers.

Applications of GHR-106-related CAR-T technology in cancer immunotherapy can be achieved by genetically modifying appropriate immune cells, e.g. T cells, by insertion of a nucleotide vector encoding GHR-106 CAR, so that those immune cells will subsequently express a GHR-106 CAR comprising an scFv of GHR-106 that can bind to the GnRH receptor expressed on the cancer cell surface to induce apoptosis and related cytotoxic killing of tumor cells, in vitro and in vivo.

A GHR-106 CAR construct can be transduced into isolated immune cells, e.g. T cells, of a given patient. These modified immune cells, e.g. T cells, from the given patient can be expanded by in vitro culture, and then transfused to the same given patient. In some embodiments, the isolated immune cells, e.g. T cells, are obtained from a healthy subject, genetically modified to insert a nucleotide vector capable of expressing a GHR-106 CAR therein, and then introduced into the bloodstream of a patient suffering from cancer. Cancer immunotherapy using a GHR-106 CAR can be used in the treatment of cancer, for example for inhibition and/or reduction of tumor growth.

In view of the widespread expression of the human GnRH receptor on the surface of a large number of different varieties of human cancer cells, it can be expected that the GHR-106 CAR construct will have broad therapeutic applications to many forms of human cancers which have an associated high level of expression of the human GnRH receptor.

With reference to the figures, a specific example embodiment of a GHR-106 CAR is now described. In some embodiments, the antigen binding domain of the GHR-106 CAR comprises a peptide that binds to the extracellular domains of the GnRH receptor in a manner similar to the GHR-106 antibody. U.S. Pat. No. 9,273,138 to Lee discloses the nucleotide sequence of humanized GHR-106 antibody. In that reference, the sequence was verified by repeated sequencing and molecular biological analysis. The antibody-producing stable cell line for a humanized GHR-106 antibody was established and disclosed by that reference.

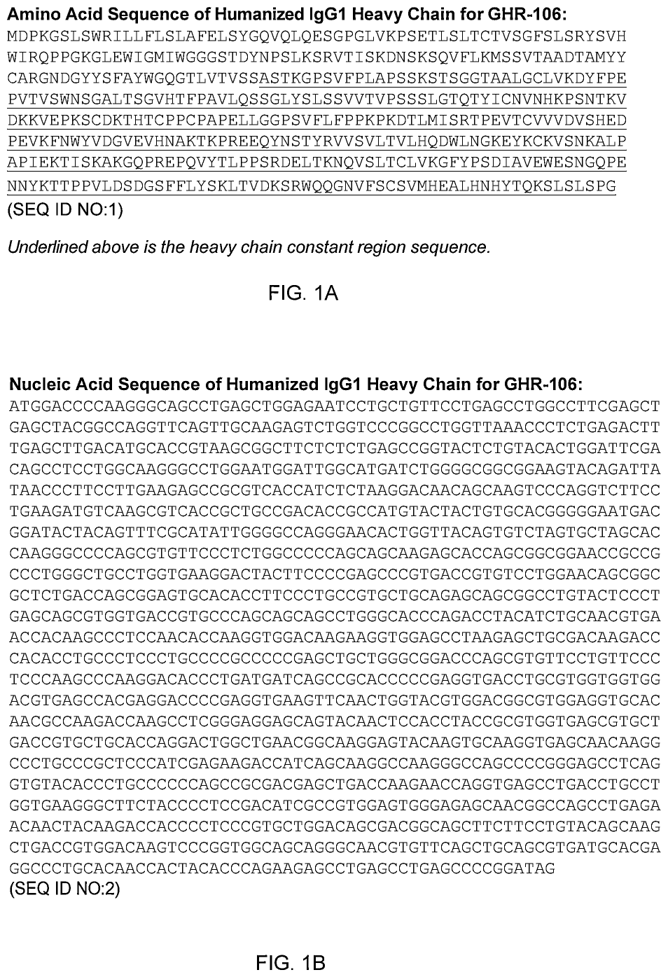

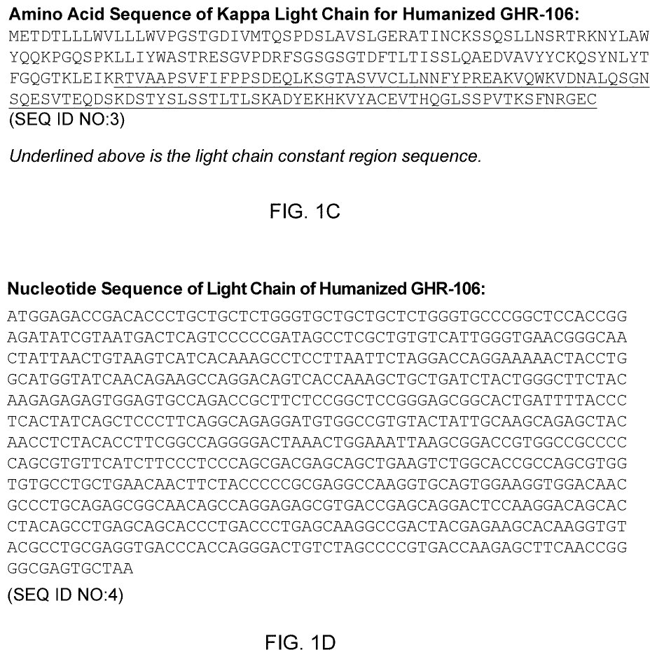

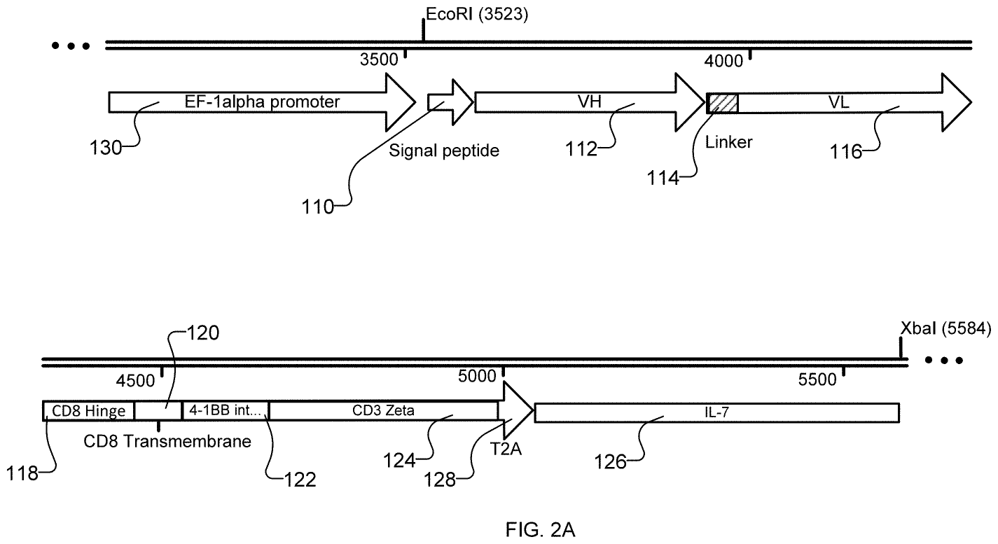

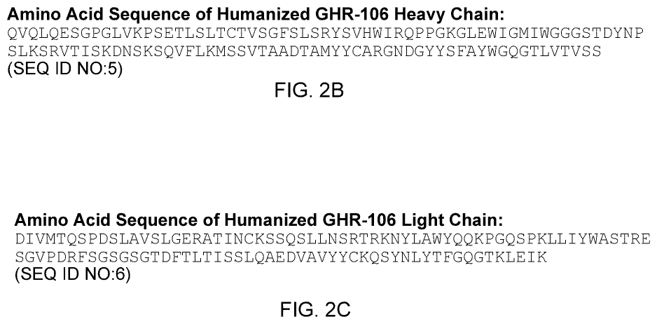

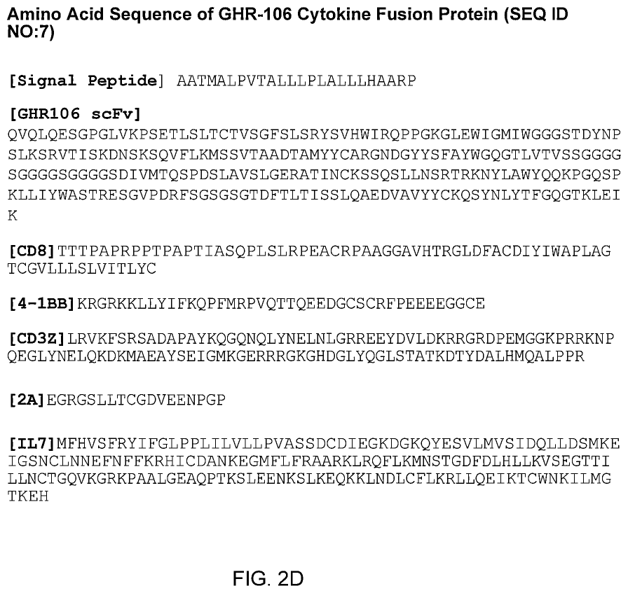

The amino acid and nucleotide sequences of an example embodiment of a humanized GHR-106 monoclonal antibody are shown in FIGS. 1A-1D. The heavy chain of the humanized GHR-106 antibody is encoded by the nucleotide sequence shown in FIG. 1B (SEQ ID NO:2). The light chain of the humanized GHR-106 antibody is encoded by the nucleotide sequence shown in FIG. 1D (SEQ ID NO:4). FIGS. 1A and 1C show the corresponding amino acid sequences of the heavy and light chains, respectively, of the humanized GHR-106 antibody (SEQ ID NOS:1 and 3, respectively). FIG. 2B shows the amino acid sequence of the heavy chain (V_{H}) of the scFv of the humanized GHR-106 antibody (SEQ ID NO:5), and FIG. 2C shows the amino acid sequence of the light chain (V_{L}) of the scFv of the humanized GHR-106 antibody (SEQ ID NO:6).

With reference to FIG. 2A, the partial structure of an example embodiment of a nucleotide vector encoding a GHR-106 CAR is illustrated. In the example embodiment, the GHR-106 CAR comprises from N-terminal to C-terminal a signal peptide 110, a V_{H} fragment of a GHR-106 monoclonal antibody 112, a linker 114, a V_{I—} fragment of a GHR-106 monoclonal antibody 116, a hinge region 118, a transmembrane domain 120, a costimulatory domain 122, and a CD3 zeta subunit domain 124.

The signal peptide 110 is used to direct the translated GHR-106 CAR into the endoplasmic reticulum, so that the antigen binding domain of the GHR-106 CAR will be expressed on the surface of an immune cell. In the illustrated embodiment, the signal peptide 110 comprises the interleukin 2 signaling sequence (IL2ss), which is used to direct the CAR for cell membrane expression in an immune cell. In alternative embodiments, any signaling domain that directs the CAR to be appropriately expressed in a membrane of an immune cell could be used.

The antigen binding domain in the exemplary embodiment of FIG. 2A comprises the V_{H} region 112, linker 114 and V_{L} region 116 that together are an scFv of a GHR-106 monoclonal antibody. In the exemplary embodiment described and characterized herein, the antigen binding domain of the GHR-106 CAR comprises the scFv of a humanized GHR-106 antibody, having the V_{H} and V_{L} regions of the humanized GHR-106 antibody having SEQ ID NOS:5 and 6, respectively shown in FIGS. 2B and 2C, joined by a peptide linker.

In alternative embodiments, the sequences of the V_{H} and V_{L} regions of antigen binding fragment of the GHR-106 CAR (i.e. the scFv of GHR-106) could be the V_{H} and V_{L} regions, respectively, of any other humanized GHR-106 antibody.

In alternative embodiments, any suitable peptide linker sequence could be used to join the V_{H} and V_{L} regions of the scFV of the GHR-106 antibody in the GHR-106 CAR. Some parameters limiting the nature of a linker used in an scFV are that it be sufficiently soluble and that it allow the V_{H} and V_{L} regions of the antibody to bind to the target antigen.

In example embodiments, the linker used in the scFV of the GHR-106 antibody can be between about 10 and about 25 amino acids in length, including any value therebetween e.g. 11, 12, 13, 14, 15, 16, 17, 18, 19, 20, 21, 22, 23 or 24 amino acids in length. In some embodiments, the linker used in the scFV of the GHR-106 antibody is rich in glycine. In some embodiments, the linker used in the scFV of the GHR-106 antibody includes a plurality of serine and/or threonine residues, to enhance the solubility of the linker.

While in the illustrated embodiment, the linker region of the scFv joins the C-terminus of the V_{H} portion of the GHR-106 antibody with the N-terminus of the V_{L} portion of the GHR-106 antibody, in alternative embodiments, the linker region of the scFv could join the C-terminus of the V_{L} portion of the GHR-106 antibody with the N-terminus of the V_{H} portion of the GHR-106 antibody.

The antigen binding domain of the GHR-106 CAR enables immune cells that have been genetically engineered with a nucleotide vector encoding the GHR-106 CAR to specifically bind to the extracellular domains of human GnRH receptor, which is expressed in cancer cells.

In some embodiments, the GHR-106 CAR binds to an epitope in the N-terminal amino acid positions 1-29 of the human GnRH receptor. In some embodiments, the antigen binding domain of the GHR-106 CAR has a specificity and affinity for binding extracellular domains of the human GnRH receptor that is comparable to murine GHR-106. In some embodiments, the antigen binding domain of the GHR-106 CAR has a specificity and affinity for binding extracellular domains of the human GnRH receptor that is comparable to humanized GHR-106.

In some embodiments, the antigen binding domain of the GHR-106 CAR has a binding affinity for the human GnRH receptor associated with a dissociation constant (K_{D}) of at least 10⁻⁷ M, 10⁻⁸ M10⁻⁹ M or 10⁻¹⁰ M, or any value within that range.

In the illustrated embodiment, a hinge region 118 is provided that extends between the antigen binding domain of the GHR-106 CAR (110, 112, 114) and the transmembrane domain 120. Hinge region 118 ensures that the antigen binding domain (i.e. the scFv of GHR-106 in the illustrated embodiment) is free to bind to the extracellular domains of the GnRH receptor in vivo. In the illustrated embodiment, hinge region 118 comprises the hinge domain of a CD8 molecule. In alternative embodiments, any suitable hinge region that allows the antigen binding domain (e.g. the scFv of GHR-106) of the GHR-106 CAR to bind to the extracellular domains of the GnRH receptor could be used.

In the illustrated embodiment, the transmembrane domain from a CD8 molecule is used to provide transmembrane domain 120. In alternative embodiments, any suitable transmembrane domain that allows the antigen binding domain and the intracytoplasmic domain of the GHR-106 CAR to be coupled together and extend through the cell membrane of an immune cell could be used.

In the illustrated embodiment, the GHR-106 CAR comprises a 4-1 BB costimulatory domain 122 present on the intracellular portion of the protein. Without being bound by theory, it is believed that the 4-1 BB costimulatory domain costimulates T cells to improve CAR-T persistence in vivo. In some embodiments, the 4-1 BB costimulatory domain could be omitted. In some embodiments, the 4-1BB costimulatory domain could be replaced by a different domain that improves CAR-T persistence in vivo.

In the illustrated embodiment, the CD3 zeta subunit domain 124 performs the function of signaling within T-cells. Without being bound by theory, once the GnRH receptor is bound by the antigen binding domain of the GHR-106 CAR, the CD3 zeta subunit domain transmits an activation signal to the T-cell, to initiate killing of the cell expressing the GnRH receptor.

Some embodiments of the invention provide a nucleotide vector for the introduction and expression of humanized GHR-106 monoclonal antibody or a fragment thereof in immune cells, such as T cells, to confer binding specificity for the extracellular domains of the human GnRH receptor on that immune cell. The nucleotide vector comprises a nucleic acid molecule encoding a GHR-106 CAR construct. In some embodiments, the vector is a vector suitable for transduction into a target cell, such as a T cell. In some embodiments, the vector is a vector suitable to be introduced into a target cell, such as an immune cell and including a T cell, by any available means of genetic engineering.

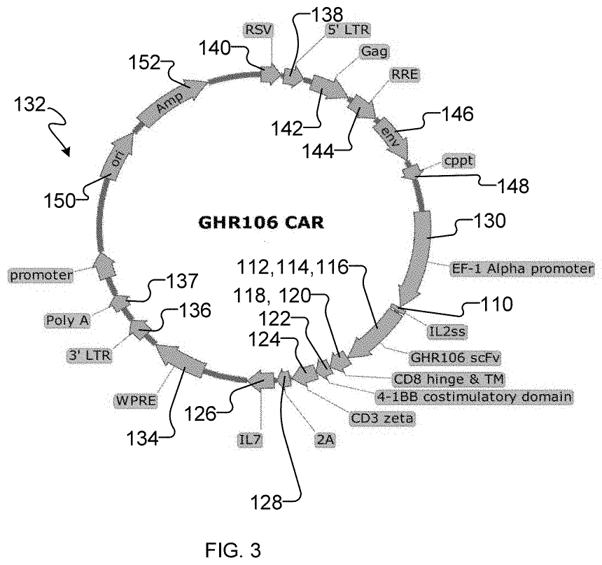

In one example embodiment, the nucleotide vector comprises a lentiviral vector encoding a GHR-106 CAR construct as shown in FIG. 3. The exemplary nucleotide vector construct encodes a polypeptide having the amino acid sequence of SEQ ID NO:7. The polypeptide having the amino acid sequence of SEQ ID NO:7 is a fusion protein comprising a GHR-106 CAR and a cytokine 126, separated by a self-cleavable peptide 128 so that the two proteins can be separated after translation.

In some embodiments, the nucleotide vector provides for expression of a GHR-106 CAR in a T cell after that T cell has been transduced with a suitable nucleotide vector construct that is capable of expressing a GHR-106 CAR.

In some embodiments, a nucleotide vector capable of expressing a GHR-106 CAR in a suitable host cell is provided. In some embodiments, the nucleotide vector is provided as a lentiviral vector. FIG. 2A shows schematically a portion of the nucleotide sequence of an exemplary GHR-106 CAR lentiviral vector, which is an example of a nucleotide vector capable of expressing a GHR-106 CAR. FIG. 3 shows schematically the full structure of a nucleotide vector capable of expressing a GHR-106 CAR in a suitable host cell, in which the nucleotide vector comprises a transfer plasmid for use in a lentiviral gene therapy system.

In some embodiments, the nucleotide vector capable of expressing a GHR-106 CAR comprises nucleic acid sequences encoding the signal peptide 110, V_{H} fragment 112, linker 114, V_{L} fragment 116, hinge region 118, transmembrane domain 120, costimulatory domain 122, and CD3 zeta subunit domain 124 described above.

In some embodiments, with reference to FIG. 2A, the nucleotide vector capable of expressing a GHR-106 CAR further encodes a cytokine 126, which without being bound by theory may be important for T cell development. In some embodiments, the nucleotide vector encodes the cytokine 126 so that it will be expressed as a C-terminal fusion protein with the GHR-106 CAR. In some such embodiments, the nucleotide vector capable of expressing a GHR-106 CAR further encodes a self-cleavage peptide 128 that interposes the GHR-106 CAR and the cytokine 126, so that after translation thereof, the GHR-106 CAR will self-cleave from the cytokine 126. A self-cleavage peptide sequence allows the expression of two proteins from the same RNA. After translation, the peptide containing the two proteins will self-cleave at the self-cleavable peptide sequence region.

In some embodiments, the cytokine 126 is IL-7, or another interleukin such as IL-15. In alternative embodiments, any desired cytokine could be used, or additional cytokines separated by additional self-cleavage peptides could be used.

In the illustrated embodiment, the self-cleavage peptide 128 comprises 2A. In alternative embodiments, any suitable self-cleaving peptide sequence could be used.

In some embodiments, the nucleotide vector capable of expressing a GHR-106 CAR comprises a promoter sequence 130, to drive expression of the GHR-106 CAR in vivo. In the illustrated embodiment of FIG. 2A, the promoter is the EF-1 alpha promoter. In alternative embodiments, any suitable promoter can be used.

With reference to FIG. 3 in which like reference numerals refer to like elements of FIG. 2A, additional elements present on an example embodiment of a nucleotide vector capable of expressing a GHR-106 CAR are shown. In the illustrated example embodiment of FIG. 3, the nucleotide vector capable of expressing a GHR-106 CAR comprises a lentiviral plasmid 132. Lentiviral plasmid 132 is a transfer plasmid that can be used to transfect eukaryotic cells to produce viruses bearing the nucleotide sequences encoding the GHR-106 CAR, which can in turn be used to carry out genetic engineering of suitable immune cells of a subject, for example T cells, to produce immune cells that express the GHR-106 CAR.

In some embodiments, a post-transcriptional regulatory element is provided on the nucleotide vector. In the illustrated embodiment, the post-transcriptional regulatory element is Woodchuck hepatitis virus post-transcriptional regulatory element 134, which stimulates the expression of transgenes via increased nuclear export. In some embodiments, any suitable post-transcriptional regulatory element can be used.

In some embodiments, a 3′ LTR 136 is provided on the nucleotide vector capable of expressing a GHR-106 CAR, to terminate transcription by the addition of a poly-A tract 137 just after the R sequence.

In some embodiments, a 5′ LTR 138 is provided on the nucleotide vector capable of expressing a GHR-106 CAR, to act as a promoter for RNA polymerase II.

In some embodiments, the nucleotide vector capable of expressing a GHR-106 CAR contains a transcription promoter. In the illustrated embodiment, the transcription promoter is a constitutive promoter. In the illustrated embodiment, the transcription promoter is a Rous Sarcoma Virus (RSV) constitutive promoter 140.

In some embodiments, the nucleotide vector capable of expressing a GHR-106 CAR contains a Gag sequence 142. Gag is a precursor structural protein of the lentiviral particle containing the matrix, capsid and nucleocapsid components.

In some embodiments, the nucleotide vector capable of expressing a GHR-106 CAR contains a Rev Response Element (RRE) 144, which is a sequence to which the Rev protein binds.

In some embodiments, the nucleotide vector capable of expressing a GHR-106 CAR contains a gene encoding VSV-G envelope protein (indicated as “env” 146).

In some embodiments, the nucleotide vector capable of expressing a GHR-106 CAR contains a central polypurine tract (cppt, 148), which is a recognition site for proviral DNA synthesis that increases transduction efficiency and transgene expression.

In some embodiments, the nucleotide vector capable of expressing a GHR-106 CAR contains an origin of replication (ori, 150) to allow for replication of the plasmid.

In some embodiments, the nucleotide vector capable of expressing a GHR-106 CAR contains an antibiotic resistance marker, which in the illustrated embodiment is an ampicillin resistance marker, Amp 152.

In some embodiments, an immune cell expressing a GHR-106 CAR is provided. In some embodiments, a GHR-106 CAR is expressed on the surface of a suitable immune cell, e.g. a T cell or a natural killer, NK, cell.

In some embodiments, a GHR-106 CAR is present on the surface of a T cell or an NK cell and the GHR-106 CAR binds to the human GnRH receptor expressed on the surface of cancer cells. In some embodiments, the GHR-106 CAR binds to human GnRH receptor expressed on the surface of cells with a specificity and an affinity comparable to that of humanized GHR-106 antibody. The GHR-106 CAR binds to human GnRH receptor on cancer cells, thereby mediating killing of the cancer cells by the T cell or NK cell.

In some embodiments, lentivirus bearing the nucleotide vector capable of expressing a GHR-106 CAR are used to carry out gene therapy. Suitable immune cells, for example T cells, are harvested from either a cancer patient (for autologous CAR-T therapy) or from a healthy subject (for allogenic CAR-T therapy). The T cells are transduced with the nucleotide vector capable of expressing the GHR-106 CAR via a lentiviral vector. The genetically engineered T cells capable of expressing the GHR-106 CAR are then introduced into the body of the cancer patient to selectively kill cancer cells expressing the human GnRH receptor.

In some embodiments, the nucleotide vector capable of expressing a GHR-106 CAR is a lentiviral vector, and the lentiviral vector is introduced into the T cells as RNA via a lentivirus vector. Once inside the T cells, the RNA is reverse-transcribed to yield DNA, which integrates with the genome of the T cell via the viral integrase enzyme.

Some embodiments of the present invention are directed to a genetically modified T cell capable of expressing a GHR-106 CAR. In some embodiments, the genetically modified T cell is obtained via transduction with a nucleotide vector capable of expressing a GHR-106 CAR. In some embodiments, the genetically modified T cell is produced by any suitable genetic modification technique, e.g. gene editing using clustered regularly interspaced short palindromic repeats (“CRISPR”)/Cas9 technology can be carried out, so that the genetically modified T cell will produce a GHR-106 CAR. This will cause the immune cell to express GHR-106 CAR incorporating an antigen-binding fragment of GHR-106 that binds to the extracellular domains of human GnRH receptor. In some embodiments, the immune cells are T cells or natural killer (NK) cells.

Some embodiments relate to methods of treating cancer. In one example embodiment, a method of treating cancer comprises: i) genetically modifying T cells obtained from a subject with suitable nucleotide vectors encoding a GHR-106 CAR construct; and ii) introducing the genetically modified T cells into the patient suffering from cancer. In some embodiments, the T cells are obtained from the patient suffering from cancer. In some embodiments, the T cells are obtained from a healthy subject, genetically modified, and then introduced into a patient suffering form cancer.

Upon transduction or genetic engineering to introduce a nucleotide vector capable of expressing a GHR-106 CAR into immune cells such as T cells or NK cells, the genetically modified immune cells will then express the scFv chain of humanized GHR-106 antibody on their surface, which acts as an antigen binding domain. This immunoglobulin chain will bind to the human GnRH receptor expressed on the surface of cancer cells, and will result in cytotoxic killing of cancer cells via the T cells. Therefore, the GHR-106 CAR-T system can be utilized for therapeutic applications for treatment of some human cancers.

In some embodiments, a plurality of immune cells, e.g. T cells or NK cells, that express a GHR-106 CAR construct can be isolated and stored in a frozen state, e.g. at −80° C. Such cells can then be thawed at a future date for introduction into a patient who has a subsequent relapse of cancer during his or her lifetime.

In view of the widespread expression of the GnRH receptor on the surface of many human cancer cells, it can be soundly predicted that the GHR-106 CAR construct has broad potential therapeutic applications in all human cancers associated with a high level of expression of the human GnRH receptor. The type I GnRH receptor has been found to be overexpressed in cell lines derived from glioblastoma, lymphoma, leukemia, melanoma and neuroblastoma, and some embodiments of the invention may also be used for the treatment of these cancers. U.S. Pat. No. 8,163,283 to Lee tested more than 30 different human cancer cell lines and found that all tested cells, except Jurkat cells (T-cell leukemia), showed expression of GnRH receptor. Specifically, Lee tested cancer cell lines from: kidney (FS293), lung (A549, Calu-6, H441, MRC-5, WI-38), lymphoma (HEL1), leukemia (K-562), melanoma (MMAN, MMRU), neuroblastoma (SH-SYSY), human ovarian (SK-OV-3, OC-3-VGH, OVCAR-3), placenta (JEG-3, Bewo), prostate (DU145, PC-3) and T-cell leukemia (Jurkat). Schally et al. (Biol. Reprod. 2005, 73 (5):851-859), which is incorporated by reference herein, found that the human GnRH receptor was expressed in a significant proportion of breast, ovarian, endometrial, prostate, renal, and pancreatic cancers.

In some embodiments, the cancer is glioblastoma, lymphoma, leukemia, melanoma, neuroblastoma, or cancer of the colon, liver, kidney, lung, breast, ovary, cervix, endometrial tissue, placenta, prostate, or pancreas.

In some embodiments, the cancer is a hormone-sensitive cancer.

While the exemplary embodiments described herein have been described with reference to human GnRH receptor, similar embodiments with appropriate modifications could be used in other mammals that suffer from cancers involving expression of the GnRH receptor on the surface of the cancer cell.

While in one exemplary embodiment described herein a lentiviral vector system has been described for use in the transduction of immune cells to express a GHR-106 CAR, in alternative embodiments, any suitable retroviral vector system could be used to carry out such transduction.

### EXAMPLES

Specific embodiments of the invention are described with reference to the following examples, which are intended to be illustrative and not limiting in nature.

### Example 1.0

### Preparation of Nucleotide Vector Capable of Expressing GHR-106—Target Plasmid

Using the sequences of the V_{H} and V_{L} portions of the humanized GHR-106 monoclonal antibody, the full length of GHR-106 CAR nucleotide cassette construct is synthesized according to the established frame and scheme, and then sub-cloned into a lenti-Puro vector transfer plasmid using standard molecular biology techniques. The insert was confirmed by Sanger sequencing.

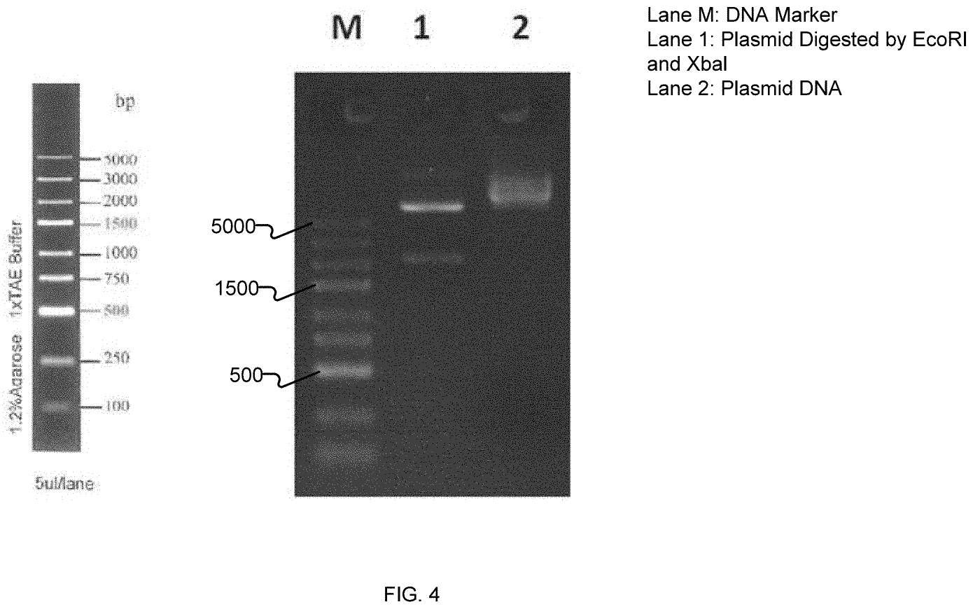

The resulting construct is validated by endonuclease digestion, as shown in FIG. 4. The recombinant vector was digested with EcoRI-XbaI, yielding the expected 2079 bp fragment.

### Example 2.0

### Preparation of Lentivirus Containing GHR-106 Transfer Plasmid

HEK293T cells (human embryonic kidney cells 293) are transfected to produce lentiviruses suitable for use in the genetic engineering of T cells to produce GHR-106 CAR. HEK293T cells are cultured overnight in complete culture medium, and are transfected with the GHR-106 transfer plasmid (plasmid 132), along with packaging plasmids including pGP (encoding Gag and Pol) and pVSVG envelope plasmid (encoding Env, VSV-G) to form lentiviral vector particles. The DNA is mixed with polyethylenimine (PEI) and then cultured with the cells. After 48 hours, supernatant is harvested and filtered to produce the virus stock, which can be aliquoted and stored at −80° C.

### Example 3.0

### Lentivirus Titration

Lentiviral copy number is determined (see e.g. Barczak et al., Mol. Biotechnol., 2015, 57:195-200, which is hereby incorporated by reference herein). HT1080 cells (a fibrosarcoma cell line) are grown, and serial dilutions of concentrated lentivirus are added to the cells together with Polybrene (hexadimethrine bromide). Virus and cells are incubated for 96 hours, then cells are washed with PBS. Genomic DNA is extracted using a Genomic DNA Purification Kit from Lifetech, and its concentration determined by NanoDrop 2000.

A standard curve for WPRE (woodchuck hepatitis virus post-transcriptional regulatory element), used as the lentiviral-specific gene, and ALB (albumin), used as a single copy reference gene, is prepared for real-time qPCR using pUC-WPRE and pUC-ALB. PCR is carried out for 40 cycles.

The primers used for PCR and detection were as follows:

| | | Fluorescent |
|---|---|---|
| Primers | 5′-3′ | group |
| WPRE_forward | GGCACTGACAATTCCGTGGT | N.A. |
| | (SEQ ID NO: 8) | |
| WPRE_reverse | AGGGACGTAGCAGAAGGACG | N.A. |
| | (SEQ ID NO: 9) | |
| WPRE_probe | ACGTCCTTTCCATGGCTGCTCGC | 5′-FAM- |
| | (SEQ ID NO: 10) | BHQ1-3′ |
| Alb_forward | GCTGTCATCTCTTGTGGGCTGT | N.A. |
| | (SEQ ID NO: 11) | |
| Alb_reverse | ACTCATGGGAGCTGCTGGTTC | N.A. |
| | (SEQ ID NO: 12) | |
| Alb_probe | CCTGTCATGCCCACACAAATCTCTCC | 5′-FAM- |
| | (SEQ ID NO: 13) | BHQ1-3′ |

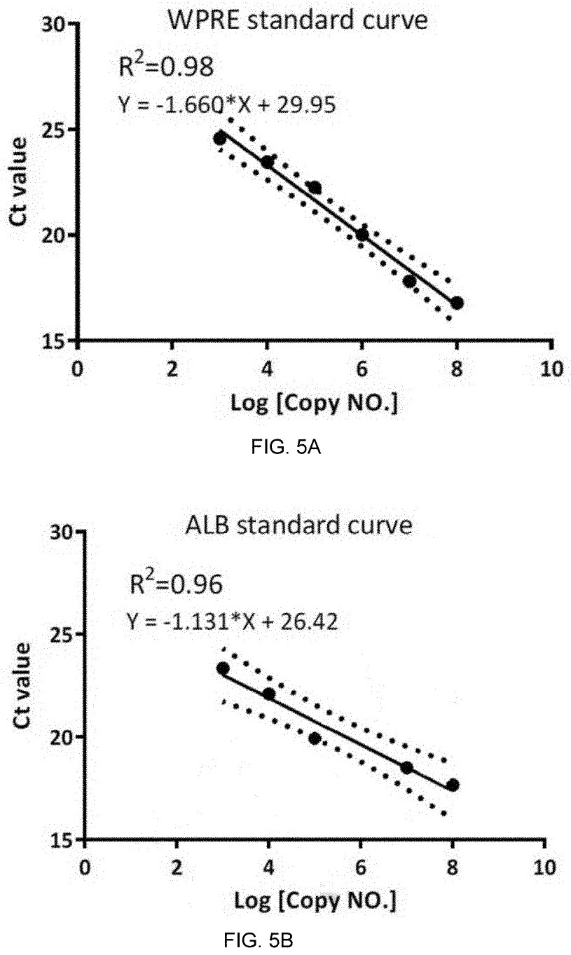

The standard curves of Ct value (cycle threshold) versus copy number obtained for WPRE and ALB are presented in FIGS. 5A and 5B. Results for the Ct value of the virus are presented in Table 1.

TABLE 1
Ct value of the virus.

| | |
|---|---|
| WPRE | ALB |

| | | | | |
|---|---|---|---|---|
| GHR-106 | 22.21 | 22.3 | 22.09 | 22.12 |

The virus titer calculation is done using the following formula:

$$Lentivirus Titer TU / mL = \frac{( CopyWPRE / CopyALB * 2 ) * Cell No .}{Volume of virus}$$

and was determined to be 4.36×10⁸ TU/mL.

This example demonstrates that lentiviral vectors were prepared. The results of lentiviral titration indicated that a lentiviral vector bearing the GHR-106 CAR transfer plasmid was successfully constructed with a high titer.

### Example 4.0

### Isolation and Preparation of Primary GHR-106 CAR T-Cells

Lymphoprep™ density gradient medium is used to separate PBMC (peripheral blood mononuclear cells including T cells) from other components of whole blood samples. Magnetic Dynabeads™ CD3 are used to isolate CD3⁺ T cells, and resulting cells are washed with PBS and resuspended and cultured in X-vivo 15 medium.

Lentiviral vector particles were used to transduce the T cells, with an MOI (multiplicity of infection) of 20. Lentiviral vector particles are defrosted and mixed with Polybrene (hexadimethrine bromide) and isolated T cells. After centrifugation, the cell pellet is harvested, resuspended in fresh medium, and cells are cultured.

### Example 5.0

### Validation of Insertion of GHR-106 CAR in Transduced T Cells

RT qPCR was carried out to determine the number of copies of nucleotide vector encoding GHR-106 in lentivirus-transduced T cells. Genomic DNA is extracted from transduced T cells using a Genomic DNA Purification Kit (Lifetech). DNA concentration is determined using Nanodrop 2000.

pUC-LTR and pUC-ALB plasmids are prepared, and serial dilutions are made to prepare the standard curve for RT qPCR. Primers used for ALB are those of SEQ ID NOS:11, 12 and 13. Primers used for LTR are as follows below. PCR is carried out for 40 cycles.

| Primer | Sequence (5′-3′) | Fluorophore |
|---|---|---|
| LTR F | TGACAGCCGCCTAGCATTTC | None |
| | (SEQ ID NO: 14) | |
| LTR R | GCTCGATATCAGCAGTTCTTGAAG | None |
| | (SEQ ID NO: 15) | |
| LTR Probe | CACGTGGCCCGAGAGCTGCATC | 5′-FAM-BHQ1-3′ |
| | (SEQ ID NO: 16) | |

ALB and LTR standard curves are generated to determine copy number for GHR-106 CAR validation in transduced T cells. After obtaining the Ct value, the Copy No. of GHR-106 CAR in the resultant recombinant T cells is calculated based on the formulation below.

$$Average Copy No . / cell = \frac{{Copy}_{LTR}}{{Copy}_{ALB}} * 2$$

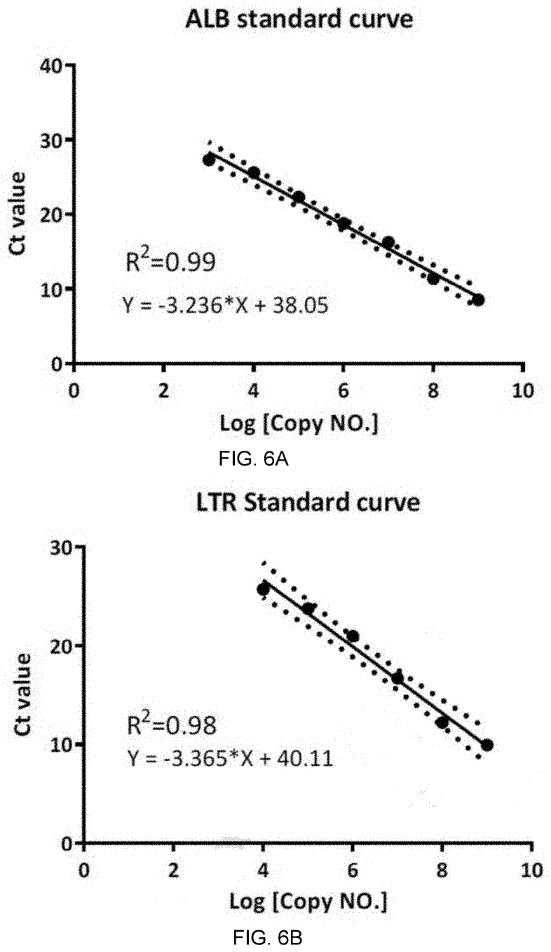

Results for the standard curves for ALB and LTR are presented in FIGS. 6A and 6B, and results for the Ct value of the samples are given in Table 2. The average number of GHR-106 CAR gene copies in the genetically modified CAR-T cells was determined to be 2.2/cell.

TABLE 2
Ct value of samples.

| | | |
|---|---|---|
| Sample | LTR | ALB |

| | | | | |
|---|---|---|---|---|
| GHR-106 CAR-T cells | 26.49 | 25.92 | 24.94 | 24.73 |

These qPCR results showed that the genes of the constructed GHR-106 CAR nucleotide vector was successfully transduced into T cells.

### Example 6.0

### Lysis of Target Tumor Cells with GHR-106 CAR-T Cells

Tumor cells from a cell line of cervical carcinoma C33A (ATCC HTB-31) were employed as target tumor cells and cultured with GHR-106 CAR-T cells at three different E/T ratios under standard cell culture conditions. C33A cells are known to express GnRH: see e.g. U.S. Pat. No. 8,163,283 to Lee.

Target C33A cells are grown to logarithmic phase, then lifted with trypsin and incubated overnight in assay wells. Prior to the assay, the assay wells are aspirated completely to remove culture and the cells are washed with sterilized PBS. GHR-106 CAR-T cells obtained in Example 4.0 are resuspended in RPMI 1640 medium without FBS and added to each assay well. Following 6 hours of co-culturing of both C33A tumor cells and GHR-106 CAR-T cells, the supernatant was harvested for determination of amount of lactate dehydrogenase (LDH) reduced using LDH detection reagent and the OD value was recorded. The percentage of target cell lysis was calculated as follows below. Maxi Lysis and Mini lysis were determined using four wells containing target C33A cells without GHR-106 CAR T cells, and cell lysis buffer was added to the Maxi lysis wells.

$$Lysis \% = \frac{( ODeach well - OD mini lysis )}{ODmaxi lysis}$$

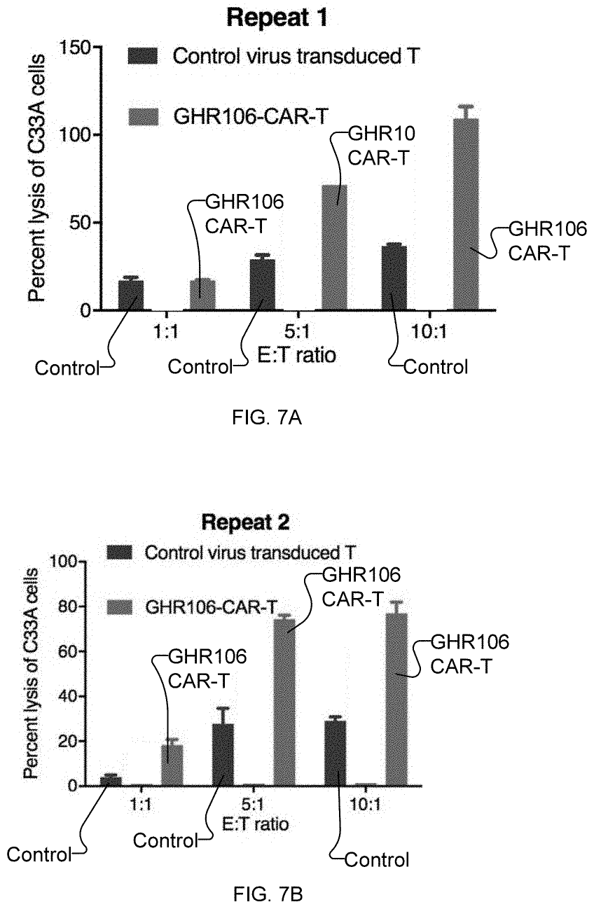

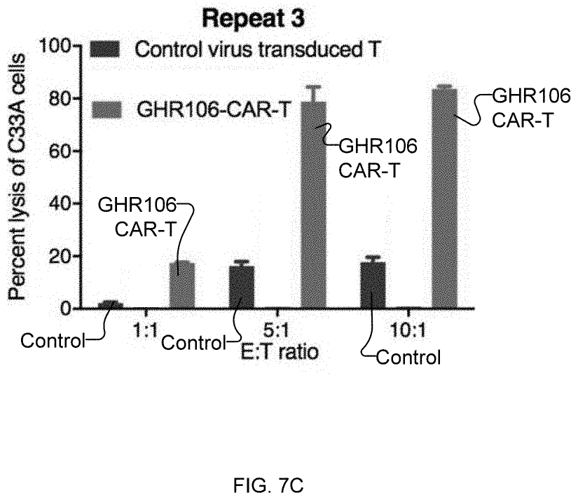

The experiments were repeated three times as shown in FIGS. 7A-7C for comparison.

The results of lysis assay strongly demonstrate that GHR-106 CAR-T cells are capable of killing the target tumor cells in a dose dependent manner. The untransduced T cells also showed a low degree of cytolytic effect compared to that of the transduced GHR-106 T cells. Without being bound by theory, this high background could potentially result from the activation of untransfected T cells by anti-CD3 and anti-CD28 antibodies in the assay system tested. The cell lytic effects of GHR-106 CAR-T cells on the tumor cells are clearly statistically significant (P 0.05-0.001).

Without being bound by theory, the results of this example demonstrate that a GHR-106 CAR present on the surface of a genetically modified T cell can mediate cytotoxicity toward a target cell. The GHR-106 binds to the GnRH receptor present on a target cell expressing the GnHR receptor and mediates killing of the target cell by the genetically modified T cell.

### Example 7.0

### Demonstration of Cytokine Release by CAR-T Cells Upon Co-Culturing With Tumor Cells Using ELISA Assay

Upon co-culturing of CAR-T cells with C33A cancer cells for 8 hours, the secretions of different cytokines were determined by typical enzyme immunoassay (EIA). These cytokines including IL-2, IL-7 and IFN-gamma were quantitatively determined and repeated three times.

Briefly, cells are adjusted to logarithmic phase. Adherent cells are lifted with trypsin, and cells are inoculated into assay wells and incubated overnight. GHR-106 CAR-T cells are harvested by centrifugation and resuspended in 1640 medium without

FBS. Target tumor cells are washed with sterilized PBS and GHR-106 CAR T-cells are added to each well and incubated at 37° C. for 6 hours. Assay plates are centrifuged and supernatant is harvested for detection of IL-2, IL-7 and IFN-gamma using an ELISA assay kit.

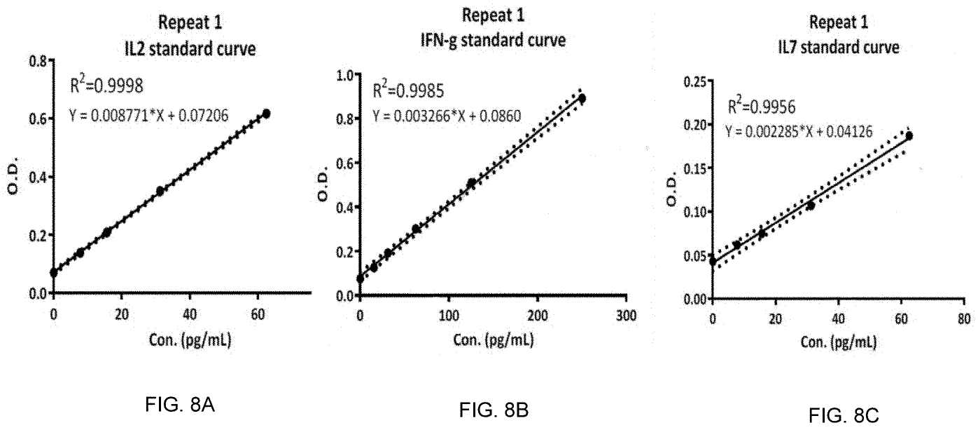

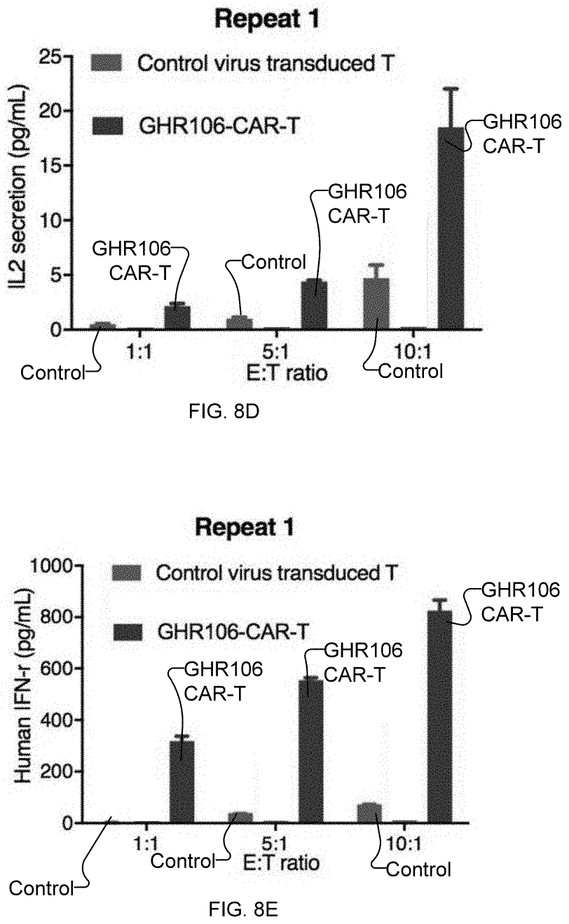

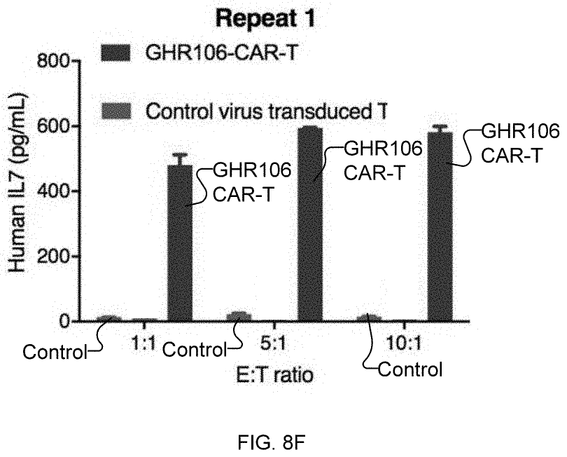

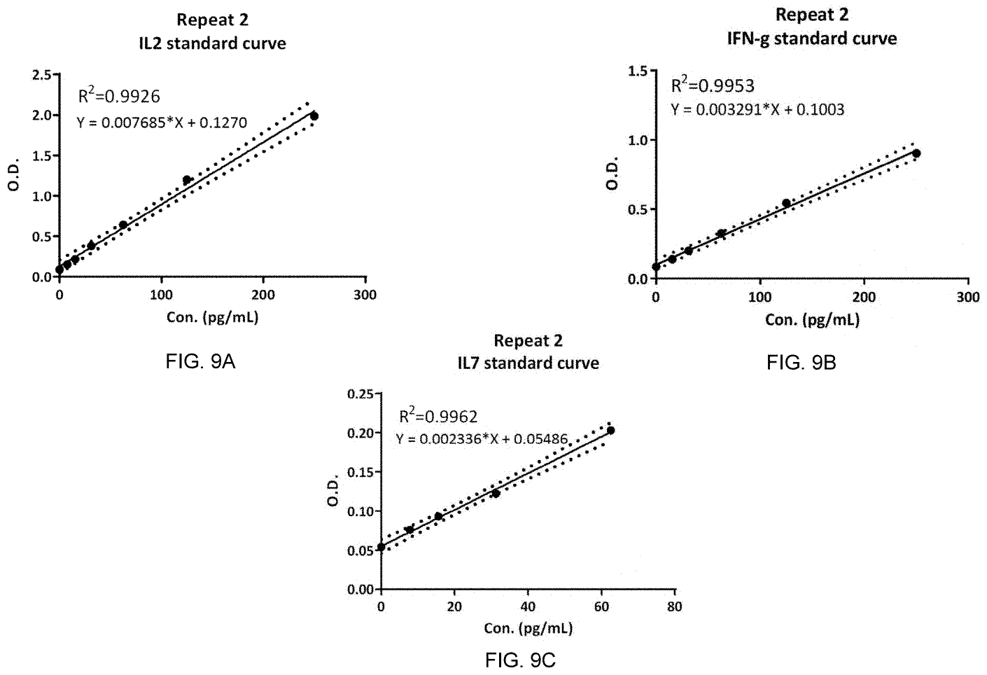

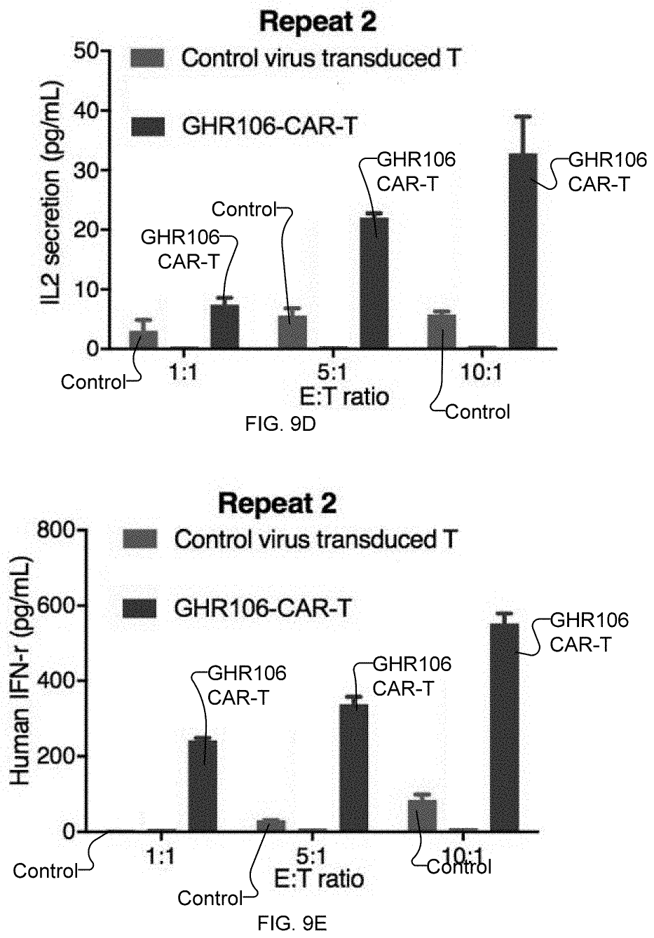

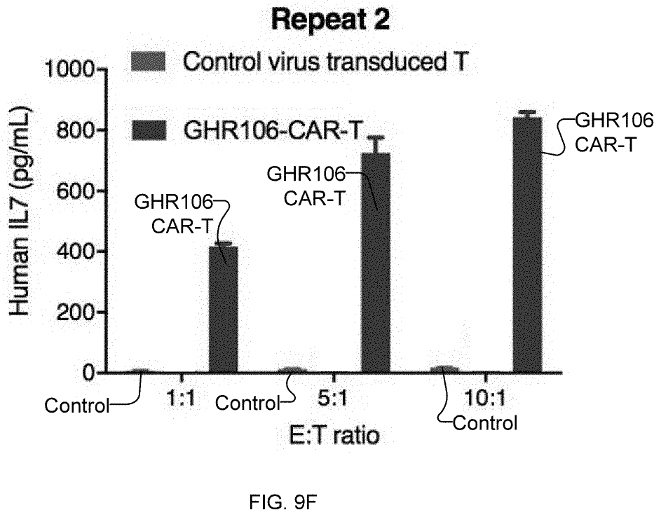

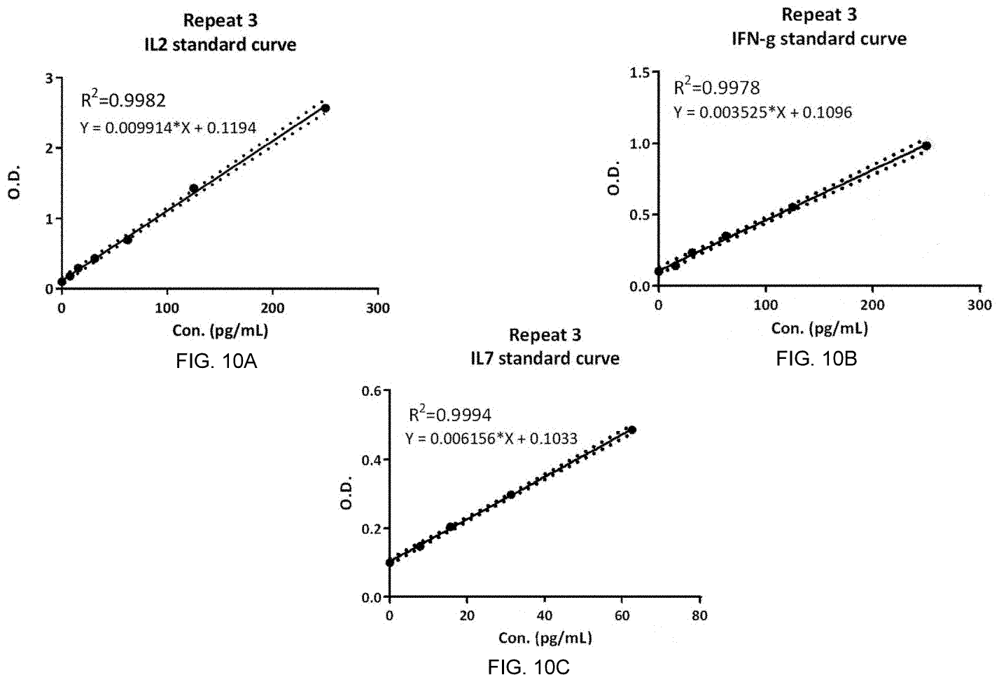

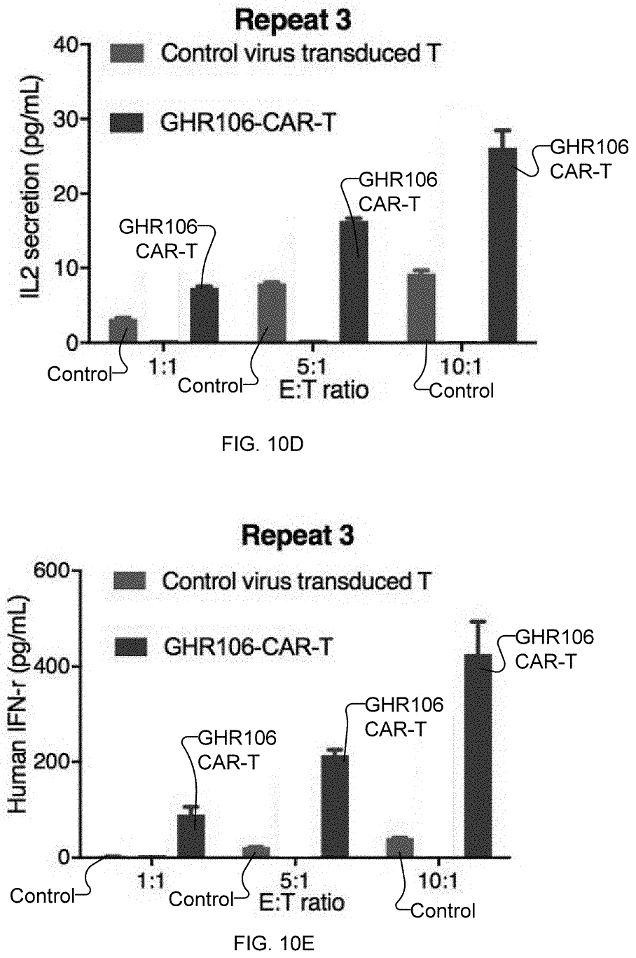

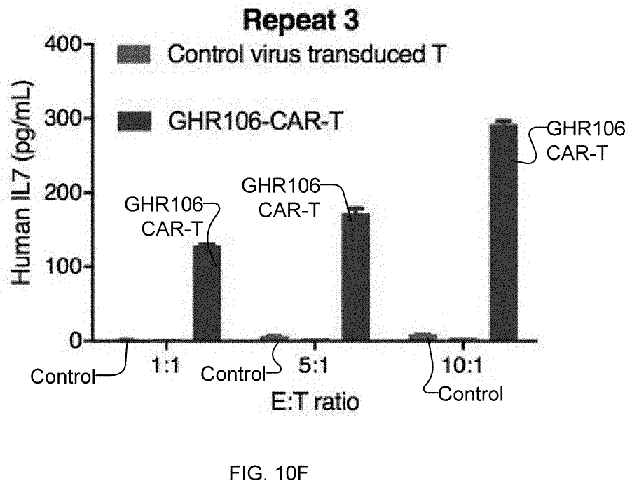

Standard curves for three different repeats of the experiment for IL-2, IFN-gamma and IL-7 are shown in FIGS. 8A, 8B, 8C, 9A, 9B, 9C and 10A, 10B, 10C, respectively.

The results of cytokine release enzyme immunoassays for three different repeats assaying IL-2, IFN-gamma and IL-7, respectively, are presented in FIGS. 8D, 8E, 8F, 9D, 9E, 9F and 10D, 10E, 10F for comparison. The cytokine release assay results suggest that significantly more cytokines were released when co-culturing C33A tumor cells with GHR-106 CAR-T cells than when co-culturing C33A tumor cells with untransfected T cells.

These examples lead to the conclusion that GHR-106 CAR-T cells (i.e. T cells transduced with a nucleotide vector capable of expressing a GHR-106 CAR) can effectively lead to cytotoxic killing of co-cultured tumor cells in vitro, and may eventually lead to significant anti-cancer efficacy in vitro or in vivo.

To summarize, the examples discussed above show the following: 1) The results of lentivirus titration showed that the inventor successfully prepared the GHR-106 CAR lentiviral vectors at a high titer. 2) The qPCR results demonstrate that the lentiviral GHR-106 CAR vector was transduced into T cells. 3) The lysis assay results demonstrate that GHR-106 CAR-T cells were able to kill target tumor cells in a “dose-dependent” manner, although untransduced T cells also showed a cytolytic effect, which without being bound by theory may result from the activation of untransduced T cells by anti-CD3 and anti-CD28 antibodies. 4) The cytokine release assay results demonstrate that GHR-106 CAR-T cells secreted more cytokines than untransduced T cells after co-incubation with target cells.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A nucleotide vector capable of expressing a GHR-106 CAR.

2. A nucleotide vector as defined in claim 1, wherein the nucleotide vector encodes a polypeptide having from N-terminal to C-terminal:
an antigen binding domain capable of binding to an extracellular domain of human GnRH receptor;
a hinge domain;
a transmembrane domain; and
an intracellular T cell signaling domain.

3. A nucleotide vector as defined in claim 2, further encoding:
a signal peptide upstream of the N-terminal portion of the antigen binding domain.

4. A nucleotide vector as defined in claim 1, wherein the antigen binding domain comprises an scFv of GHR-106.

5. A nucleotide vector as defined in claim 2 4 wherein:
the antigen binding domain comprises V_{H} and V_{L} regions of a humanized GHR-106 monoclonal antibody joined by a linker;
the signal peptide comprises the interleukin 2 signaling sequence;
the hinge domain comprises the hinge domain of a CD8 molecule;
the transmembrane domain comprises the transmembrane domain of a CD8 molecule; and/or
the intracellular T cell signaling domain comprises a CD3 zeta subunit domain.

6. A nucleotide vector as defined in claim 2, further comprising:
a costimulatory domain; and/or
a cytokine positioned at the C-terminus of the intracellular T cell signaling domain and a self-cleavable peptide sequence interposing the intracellular T-cell signaling domain and the cytokine.

7. A nucleotide vector as defined in claim 6, wherein:
the costimulatory domain comprises a 4-1BB costimulatory domain;
the cytokine comprises interleukin-7; and/or
the self-cleavable peptide sequence comprises the peptide sequence of 2A.

8. A nucleotide vector as defined in claim 2 further comprising:
a promoter positioned to drive expression of the GHR-106 CAR;
a post-transcriptional regulatory element;
a 3′ LTR;
a 5′ LTR;
a transcription promoter;
a Gag sequence;
a Rev Response Element (RRE);
a gene encoding an envelope protein (Env);
a central polypurine tract;
an origin of replication; and/or
an antibiotic resistance marker.

9. A nucleotide vector as defined in claim 8, wherein:
the promoter comprises EF-1 alpha promoter;
the post-transcriptional regulatory element comprises Woodchuck hepatitis virus post-transcriptional regulatory element;
the transcription promoter comprises a constitutive promoter, optionally Rous Sarcoma Virus (RSV) constitutive promoter; and/or
the gene encoding the envelope protein (Env) comprises a gene encoding VSV-G envelope protein.

10. A nucleotide vector as defined in claim 5, wherein the V_{H} region of the humanized GHR-106 monoclonal antibody has the amino acid sequence of SEQ ID NO:5, and/or wherein the V_{L} region of the humanized GHR-106 monoclonal antibody has the amino acid sequence of SEQ ID NO:6.

11. A nucleotide vector as defined in claim 2, wherein the antigen-binding domain of the expressed protein binds to the extracellular domains of human GnRH receptor and with an affinity substantially equivalent to a humanized GHR-106 monoclonal antibody.

12. A nucleotide vector as defined in claim 1, having the general structure shown in FIG. 3

13. A nucleotide vector as defined in claim 1 that encodes an amino acid having the sequence of SEQ ID NO:7.

14. An isolated nucleic acid molecule, comprising a nucleotide sequence encoding a polypeptide having from N-terminal to C-terminal:
a signaling domain;
an antigen binding domain capable of binding to extracellular domains of the human GnRH receptor;
a transmembrane domain;
a CD3-zeta signaling domain; and
a cytokine domain separated from the CD3-zeta signaling domain by a self-cleavable peptide.

15. (canceled)

16. A nucleotide vector as defined in claim 1, wherein the nucleotide vector comprises a lentiviral plasmid suitable for use as a transfer plasmid in a lentiviral vector system to transduce immune cells with a nucleotide sequence capable of expressing GHR-106 CAR.

17. A GHR-106 CAR comprising from N-terminal to C-terminal:
an antigen binding domain capable of binding to an extracellular domain of human GnRH receptor;
a transmembrane domain; and
an intracellular T cell signaling domain.

18. A GHR-106 CAR as defined in claim 17, further comprising
a signal peptide positioned on the N-terminal side of the antigen binding domain;
a hinge domain; and
an intracellular costimulatory domain.

19. A GHR-106 CAR as defined in claim 17, wherein:
the antigen binding domain comprises V_{H} and V_{L} regions of a humanized GHR-106 monoclonal antibody joined by a linker;
the signal peptide comprises the interleukin 2 signaling sequence;
the hinge domain comprises the hinge domain of a CD8 molecule;
the transmembrane domain comprises the transmembrane domain of a CD8 molecule;
the intracellular T cell signaling domain comprises a CD3 zeta subunit domain; and/or
the intracellular costimulatory domain comprises a 4-1BB costimulatory domain.

20. A polypeptide encoded by a nucleotide vector as defined in claim 1, the polypeptide having the amino acid sequence of SEQ ID NO.7.

21. (canceled)

22. (canceled)

23. An immune cell comprising a nucleotide vector, polynucleotide molecule or isolated nucleic acid molecule as defined in claim 1.

24-40. (canceled)
